(19) United States
(12) Patent Application Publication
(10) Pub. No.: US 2021/0196895 A1
(43) Pub. Date: Jul. 1, 2021

(54) NEEDLE-FREE INJECTION SYSTEM

(71) Applicants: Ira Yasmin Lehmann, Duesseldorf (DE); Philip Lennart Victor Perez, Duesseldorf (DE)

(72) Inventors: Ira Yasmin Lehmann, Duesseldorf (DE); Philip Lennart Victor Perez, Duesseldorf (DE)

(21) Appl. No.: 17/055,677

(22) PCT Filed: May 9, 2019
(86) PCT No.: PCT/EP2019/061955

§ 371 Date: Nov. 16, 2020

(30) Foreign Application Priority Data
May 16, 2018 (DE) 202018102738.7
Sep. 6, 2018 (WO) PCTDE2018100760
Mar. 28, 2019 (DE) 20 2019 101 771.6

(51) Int. Cl. A61M 5/30; A61M 5/20; A61M 5/315

(52) U.S. Cl. CPC A61M 5/30; A61M 5/31581; A61M 5/31553; A61M 5/2033

(57) ABSTRACT

A needle-free injection system may include an active substance cartridge, which may have a nozzle and a plunger-actuated dosing chamber for the pressurized dispensing of an active substance. The injection device may have a housing with a receptacle for accommodating the active substance cartridge and a piston, which may be movable relative to the housing and may be designed to execute a movement for the ejection of the active substance from the active substance cartridge, where a mechanism for driving the plunger may be provided, and the plunger may have a starting position related to the mechanism, from which position the piston may be released to move by means of a trigger. The mechanism may be designed to allow the piston to return to the starting position. The dispensing quantity of the active substance may be adjustable.

### TECHNICAL FIELD

The system described herein relates to a needle-free injection system (also referred to below as “NIS”), for example, an NIS including an active substance cartridge which has a nozzle and a plunger-actuated dosing chamber for the pressurized dispensing of an active substance for injection into the intracutaneous, subcutaneous or intramuscular skin layers.

### BACKGROUND OF THE INVENTION

Without the use of a hypodermic needle, an NIS enables the injection of drugs and other active substances into the intracutaneous, subcutaneous or intramuscular skin layers. The substance to be injected is here pressed by means of high pressure through a very fine nozzle. The pressure and velocity are here so enormous, that is to say they are so high, that the substance is pressed into or through the epidermis.

NISs have recently become known from the prior art. Many injection devices which are being developed and are present on the market follow a relatively similar structure in terms of system, technology and design.

It is further known to provide a mechanism (generally a tensionable helical spring) for driving the piston, which has a starting position related to the mechanism, from which position the piston is released by means of a trigger for executing the movement. The mechanism is configured such that it enables the return of the piston into the starting position.

Also known are systems in which the receptacle of the active substance cartridge is adjustable in the longitudinal direction on the housing in relation to the piston in order to set the dispensing quantity of the active substance.

With respect to the trigger/drive, at least three variants are found. The first variant is the generation of pressure by means of a gas cartridge, the second by means of an internal pressure spring which is tensioned by an external tensioning system. The third variant is an integrated lever mechanism which tensions the pressure spring.

All three variants ultimately serve to set the piston in motion with high pressure, so that the piston is capable of pressing the liquid in the dosing chamber through the approximately 80-360 micrometer small nozzle. With respect to the dosing chamber, there exists a variant in which the dosing chamber is integrated within the system, and a variant in which the dosing chamber is screwed on externally.

The NISs that are present on the market may be found in many different constructions, for example, intended for medical purposes. In many devices, the spring is tensioned with an external adapter, which makes hygienic operation almost impossible, since the dosing chamber has to be unscrewed anew for each tensioning operation, which inevitably leads to a higher number of touching contacts and thus has a considerable effect on the sterility. Moreover, in devices which are present on the market, the injection depth for beauticians may not be regulated.

In most cases, devices currently on the market possess an external adapter. The active substance cartridges having a capacity of 0.5 ml are intended to be able to be screwed externally into the device. In devices currently on the market, only one shot per filled external cartridge can take place. The handling involves the external cartridge being filled with an active substance. In the second step, the spring of the injector is tensioned with an external adapter. In the third step, the filled cartridge is screwed into the injector. In the fourth step, if the device has an adjusting device, the injectable quantity is set on the device. An active substance quantity of up to 0.5 ml can here be set. In the fifth step, the active substance is injected with one shot. After this, the active substance cartridge has to be screwed off again, the spring of the injector has to be retensioned by the external adapter, and a new active substance cartridge is screwed in place. This procedure may be too complex for a beautician, since the beautician may be only allowed to introduce a small quantity of esthetic or cosmetic active substances into the skin.

It may be desirable to provide an NIS that eliminates the above-mentioned problems, where the maximum volume delivery is limited.

### SUMMARY OF THE INVENTION

According to embodiments of the system described herein, an NIS may be characterized in that a mechanism for tensioning the mechanism for driving the piston may be integrated, wherein the receptacle of the active substance cartridge may be adjustable in the longitudinal direction by threads in relation to the piston in order to set the dispensing quantity of the active substance, where a locking profile and an appropriately corresponding locking member may be provided, by means of which the maximum stroke length of the piston, and hence the maximum dosing quantity, may be defined, so that the maximum dosing quantity of the active substance may be markedly less than the content of the active substance cartridge. For example, the maximum dosing quantity of the active substance may amount to at most 0.1 ml and/or at most one-fifth of the capacity of the active substance cartridge. For the longitudinal adjustment of the receptacle in relation to the piston, a threaded joint may be provided on the housing, the threaded joint comprising an internal thread on the receptacle and an external thread on the housing.

The advantage of the NIS according to embodiments of the system described herein thus may be seen in the fact that, on the one hand, by virtue of the locking function, a defined dispensing quantity may be dispensed, and may be dispensed to a limited insertion depth into the upper skin layers; and one the other hand the NIS may be usable as a “multi-shot system”, in which the drive mechanism may be tensioned several times in such a way that a removal of the dosing chamber is unnecessary. A beautician may with this NIS, by repeated tensioning, inject small quantities of the active substance several times without having to install a new active substance cartridge and without having to fasten an external tensioning system to the NIS. It may be hereby possible to inject skin regions involved in a treatment operation, for instance wrinkles, several times with small quantities of an active substance, and thus achieve the intended effect of the active substance.

In comparison to known systems, in some embodiments the dispensed maximum dosing quantity may be (e.g. always) ultimately markedly less than the content of the entire active substance cartridge. The maximum dosing quantity of the active substance here depends on the structure of the NIS. It may amount to, for example, at most one-fifth of the capacity of the active substance cartridge. It may be, in particular, around 0.1 ml. This value may be very suitable for cosmetic applications such as the injection of skin wrinkles with hyaluronan. The actual quantity of the injected active substance for each injection operation may be variable. Microdoses may be delivered well below the limitation of the dispensing quantity by the locking member. This means that, in treatment with hyaluronan, for example, the optimal dose may be set for each skin type. The actually delivered dosing quantity may be varied by a factor of 20, for example, within the range 0.005-0.1 ml. In this way, the delivery quantity may be precisely adapted to the skin type and the treatment objective, where, by multiple triggering, the desired delivery quantity may be repeatedly introduced up to the delivery of the entire quantity of active substance in the active substance cartridge. In this way, a fast cosmetic treatment that is individually adaptable to the specific case may be possible.

Advantageously, a tension spring (which also may be referred to as a piston spring) may be provided as the drive for the actuating piston of the NIS. Into the NIS may also be integrated a tensioning lever, by means of which the tension spring may be tensioned several times per active substance cartridge such that the entire content may be injected in a plurality of injection operations, for example around 5 to 10 injection operations.

In an embodiment of the system described herein, the relationship between the maximum stroke length of the piston and the diameter of the dosing chamber of the active substance cartridge may be configured such that the active substance, due to the dosed dispensing quantity, reaches only the upper skin layer and not the lower tissue layers. The introduction of the active substance into the upper skin layer may be more a case of a transdermal application than a conventional injection. The confinement of the application of the active substance to the upper skin layer may be achieved, for example, by confining the maximum dosing quantity of the active substance to maximally 0.1 ml.

Hence, in the NIS in this embodiment, there may be a significant correlation between the diameter of the external dosing chamber and the maximally possible stroke length of the piston.

In an embodiment, the piston, in which the pressure may be built up by means of the spring, may have at the very most a maximum stroke length of about 5.7 mm. In combination with a commercially available dosing chamber having a diameter of about 5 mm, this corresponds to a shot volume of approximately 0.1 ml.

The particularity of the system described herein may hence be seen in the fact that there may be provided an NIS which may be optimized for legal use of cosmetics and may be approved, without risk that, in cosmetic treatments, tissue layers lying beneath the upper skin layer will be reached.

As has already emerged from the above depictions, there has previously been no method which legally authorizes the beautician to use an alternative to the traditional injection of wrinkles with hypodermic needles. Beauticians may not be allowed to work with syringes. Many beauticians even pursue the lengthy path of becoming a non-medical practitioner solely in order to subsequently be able to make injections with hypodermic needles. With the present NIS, precisely this possibility may be legally open to beauticians.

### BRIEF DESCRIPTION OF THE DRAWINGS

Further objectives, features, advantages and possible applications of the NIS according to embodiments of the system described herein emerge from the following description of illustrative embodiments with reference to the drawings. All described and/or illustrated features herein form, individually or in any chosen combination, part of the subject of the system described herein, irrespective of the summary in individual claims or their back-reference.

In the drawings:

FIG. 1 shows the NIS in side view in longitudinal section, according to embodiments of the system described herein;

FIG. 2 shows a further embodiment of the NIS of the system described herein, using an electric motor, in side view in longitudinal section;

FIG. 3 shows a further embodiment of the NIS of the system described herein in side view in longitudinal section, in which the necessary supply of force may be realized by raising of the piston;

FIG. 4 shows a further embodiment of the NIS of the system described herein in side view in longitudinal section, in which the necessary supply of force may be realized by rotation of a piston extension;

FIGS. 5a and 5b show a further embodiment of the NIS of the system described herein in three-dimensional side view, wherein a shank may be arranged separably on the housing;

FIG. 6 shows the embodiment of the NIS of the system described herein from FIGS. 5a and 5b with different receptacles for different sizes of active substance cartridges, in three-dimensional side view;

FIG. 7 shows a further embodiment of the NIS of the system described herein in longitudinal section;

FIG. 8 shows the embodiment from FIG. 7 with tensioned piston spring in longitudinally sectioned three-dimensional view, according to embodiments of the system described herein;

FIGS. 9 and 10 show enlarged views of the region of the receptacle for the active substance cartridge of the embodiment of FIGS. 7 and 8 with tensioned and slackened piston spring, according to embodiments of the system described herein;

FIG. 11 shows a further embodiment of the NIS of the system described herein in longitudinal section;

FIG. 12 shows the NIS from FIG. 11 upon tensioning of the piston spring, according to embodiments of the system described herein; and

FIG. 13 shows the NIS from FIGS. 11 and 12 with tensioned piston spring according to embodiments of the system described herein.

### DESCRIPTION OF VARIOUS EMBODIMENTS

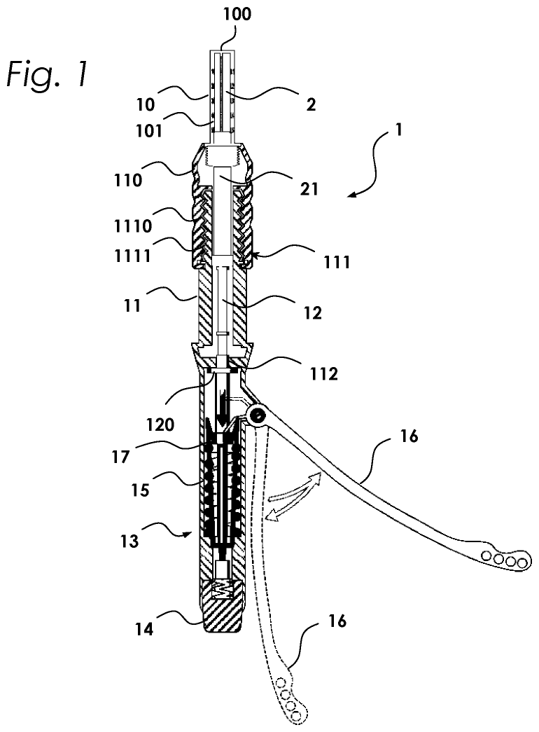

As can be seen from FIG. 1, the injection system 1 according to embodiments of the system described herein consists of an active substance cartridge 10, which may have a nozzle 100 and a plunger-actuated dosing chamber 101, for the pressurized dispensing of an active substance 2 for injection into the upper skin layer, and further may consist of a housing 11 having a receptacle 110 for the unilaterally reversible arrangement of the active substance cartridge 10. Integrated into the active substance cartridge 10 may be a movable injection piston 21, with which the active substance 2 may be forced out of the nozzle 100.

A piston 12 in the housing 11 may form an actuating piston and press against the injection plunger 21. The piston 12, which may be movable relative to the housing 11, may be configured for the pressurized ejection of the active substance 2 from the active substance cartridge 10 in a settable dosing quantity. For this purpose, the actuating piston 12 may force the injection plunger 21 into the active substance cartridge 10.

For the driving of the piston 12, a mechanism 13 in the form of a pressure spring 15 may be provided, wherein the piston 12 may have a starting position relative to the mechanism 13, from which position the piston 12 may be released by means of a trigger 14 for executing the movement. In the starting position, the piston 12 may be distanced from the active substance cartridge 10. Following the release by the trigger, the pressure spring 15 may force the piston 12 toward the active substance cartridge 10.

The mechanism 13 may be configured such that it enables the return of the piston 12 into the starting position, where the receptacle 110 of the active substance cartridge 10 may be adjustable in the longitudinal direction on the housing 11 by means of a threaded joint 111 relative to the piston 12 in order to set the dispensing quantity of the active substance 2. The threaded joint 111 may include an internal thread 1110 on the receptacle 110 and an external thread 1111 on the housing 11. By twisting of the sleeve-shaped receptacle 110 with respect to the housing 11, the distance between the active substance cartridge 10 and the piston 12 may be altered by means of the interlocking internal threads 1110 on the receptacle and external thread 1111 on the housing 11. The extent by which the piston 12 forces the injection plunger 21 into the active substance cartridge 10 may be thereby set. In this way, the dosing quantity of the active substance to be dispensed in an injection operation may be precisely set. The maximum dosing quantity may be limited by the maximum stroke length of the piston 12. In a standard active substance cartridge 10 having a diameter of 5 mm, the maximum stroke length may be limited to, for example, 5.7 mm, in order to achieve a maximum dosing quantity of approximately 0.1 ml.

On the piston 12 may be advantageously arranged a locking profile 120. On the housing 11 may be arranged an appropriately corresponding locking member 112. By means of locking profile 120 and locking member 112, the maximum stroke length of the piston 12 may be defined. The locking profile 120—as can be seen from FIG. 1—may be formed as a collar, which protrudes on the outer periphery of the piston 12. The locking member 112 may be formed as a pass-through opening for the piston 12, which opening may be arranged in the housing 11 and may have a lesser diameter than the collar 120.

The pressurization may be realized by means of a tensioning spring or piston spring 15 that spring-loads the piston 12, although the pressurization may also be realized by means of gas impingement and/or by means of an electrically powered pressurizing device, and of course, also by means of any other systems suitable for the pressurization.

In an embodiment of the system described herein, the NIS 1 comprises an integrated tensioning lever 16, by means of which the piston spring 15 may be tensioned. For the detention of the piston 12 in the starting position, a piston catch 17 may be provided. The piston catch 17 may be formed as a claw.

On the underneath of the housing 11 may be found the trigger 14, in the form of a button, for the simple release of the piston 12. With the trigger 14, the piston catch 17 may be unlocked and the actuating piston 12 situated in the starting position released. The piston spring 15 may forces the piston 12 toward the active substance cartridge 10 against the injection plunger 21, which may force the injection plunger into the active substance cartridge 10. The piston 12 may be forced, at most by the maximum stroke length of, for example, 5.7 mm, into the active substance cartridge, which itself may have an at least five times as large length of, for example, 30 mm. In some embodiments of this injection operation (shot), maximally 0.1 ml active substance may be injected. The piston spring 15 now may be retensioned with the integrated tensioning lever 16, whereupon the piston 12 may be moved by the maximum stroke length away from the active substance cartridge 10. By twisting of the sleeve-shaped receptacle 110 with respect to the housing 11, the distance between the active substance cartridge 10 and the piston 12 may be in turn reduced by the desired measure by means of the interlocking internal thread 1110 on the receptacle and external thread 1111 on the housing 11, so that the dosing quantity for the next shot may be fixed. This injection operation may be repeated until the entire active substance 2 has been ejected from the active substance cartridge 10. In such embodiments, multiple injections (may be performed in quick succession without having to change the active substance cartridge 10 or having to attach an external tensioning device to the housing 11 such that the NIS is considered a multi-shot system.

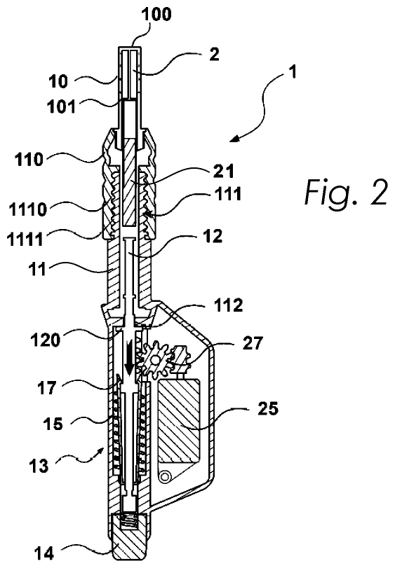

FIG. 2 shows a further embodiment of the NIS 1 according to embodiments of the system described herein, using an electric motor. In this embodiment, an electric motor 25, and perhaps a transmission system 27, may be provided to power the NIS 1, where the rotation of the electric motor 25 may be transmitted to the transmission system 27, which effects the movement of the piston 12 in a direction away from the active substance cartridge 10, which movement may create a tension in the pressure spring 15. Prior to the pressing of the trigger 14, the transmission 27 and the electric motor 25 may be advantageously decoupled so that the piston 12 may be forced by the piston spring 15 without hindrance against the injection plunger 21. Here too, the receptacle 110 may be twistable with respect to the housing 11, so that an internal thread 1110 on the receptacle and an external thread 1111 on the housing 11 enable the adjustment of the distance between piston 12 and active substance cartridge 10.

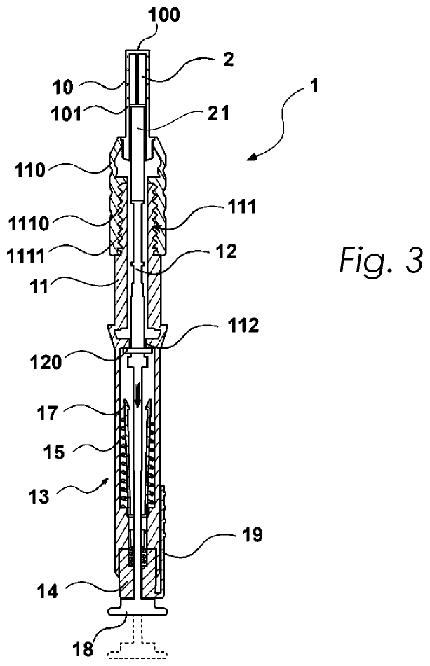

In FIG. 3 is represented a further embodiment of the NIS according to embodiments of the system described herein, in which the necessary supply of force may be realized by withdrawal of the piston 12 toward the trigger 14, whereupon the necessary tension may be generated in the pressure spring 15. A shot may be herein discharged, that is to say an injection of active substance into the skin may be triggered by downward pushing of the trigger 14 toward the active substance cartridge 10.

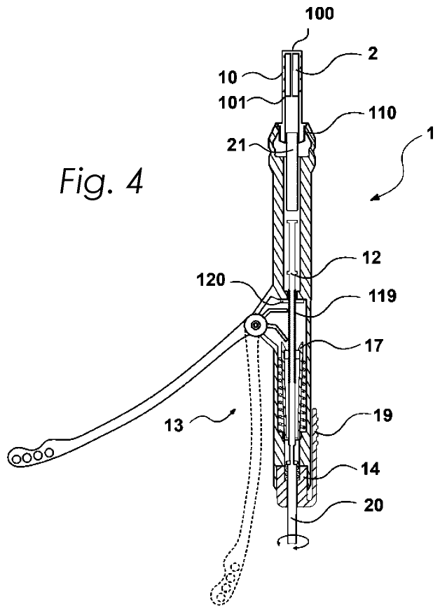

FIG. 4 shows a further embodiment of the NIS according to embodiments of the system described herein, in which the necessary supply of force may be realized by rotation of a piston extension 20. The quantity of active substance ejected from the active substance cartridge 10 for each individual shot may herein be set by altering the distance between the piston 12 and the active substance cartridge 10. This may be achieved by rotation of the piston extension 20. A threaded joint along a piston portion enables this change in distance. Calibration markings on the piston extension 20 enable the fine adjustment of the output quantity.

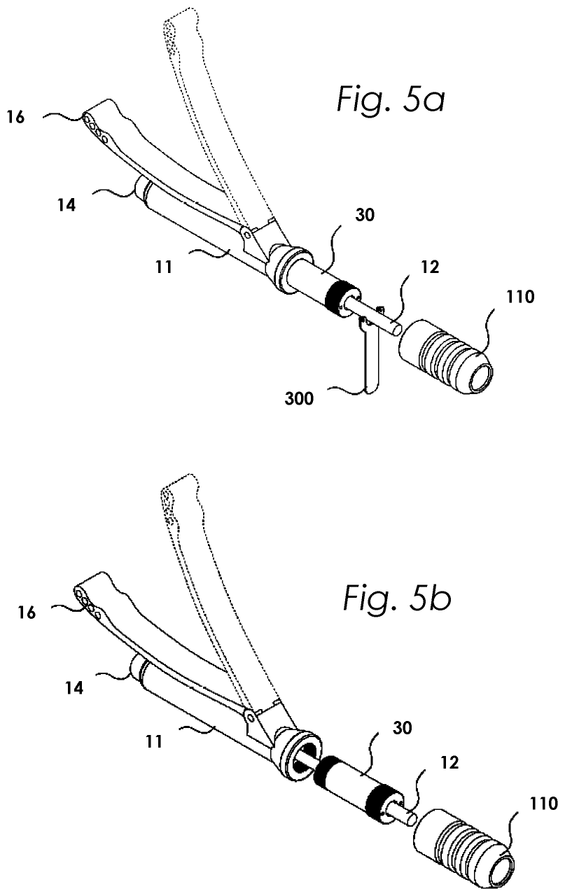

FIGS. 5a and 5b show a further embodiment of the NIS 1 according to embodiments of the system described herein, in which a shank 30 may be arranged separably on the housing 11. In order to facilitate an effective cleaning of the NIS 1, in this embodiment of the system described herein a shank 30, which may be separable from the housing 11 and may be fastened to the housing 11 via a thread on its end and a corresponding thread on a portion inside the housing 11, may be provided. The shank 30 may have the shape of a hollow sleeve through which the piston 12 passes. Since the shank 30 may be screwed tightly into the housing 11, a (e.g., special) screwing tool 300, with which the shank 30 may be screwed tightly into the housing 11 and, when necessary, unscrewed therefrom, may be provided.

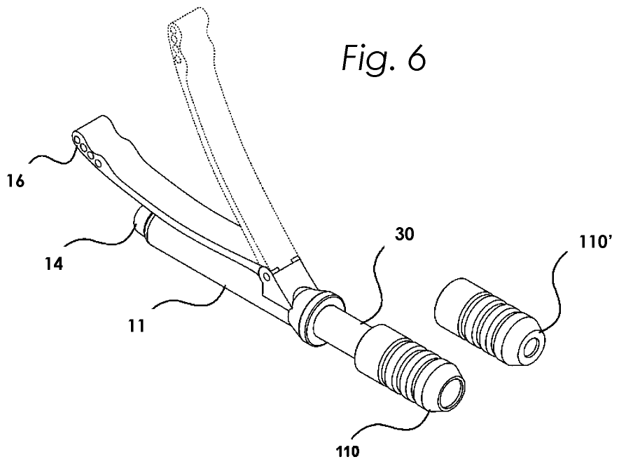

The active substance cartridge 10 for an active substance may be available in a variety of sizes. FIG. 6 shows a further embodiment of the NIS 1 according to embodiments of the system described herein with different receptacles 110 and 110′ for various sizes of active substance cartridges 10. This embodiment of the system described herein makes it possible for two sizes of active substance cartridges 10 to be arranged on the housing 11. This may be achieved by the use of two variants of the sleeve-shaped receptacles 110, 110′, into which a variant of the active substance cartridge 10 respectively may be screwed. The threaded bore at the end of each variant of the receptacles 110, 110′ advantageously corresponds to a specific cartridge size of the active substance cartridge 10. For example, cartridge sizes having a quantity of active substance of 0.3 ml and having an active substance quantity of 0.5 ml may be available. The threads on that side of each of the variants of the receptacles 110, 110′ that is facing away from the active substance cartridge may enable the fastening of the respective receptacle 110, 110′ to the shank 30, and thus the setting of the quantity of the active substance which is ejected for a single shot of the NIS 1; in that, by rotation of the sleeve-shaped receptacle 110, 110′, the distance of the thereto fastened active substance cartridge from the piston 12 may be altered.

In terms of its construction, the NIS 1 according to embodiments of the system described herein may not be confined to the above-specified embodiments. Rather, a multiplicity of design variations are conceivable, which make use of the represented solution even in a construction of fundamentally different nature.

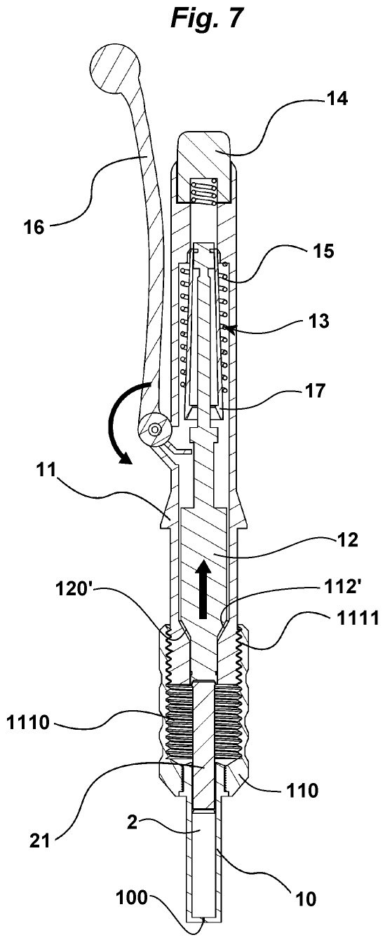

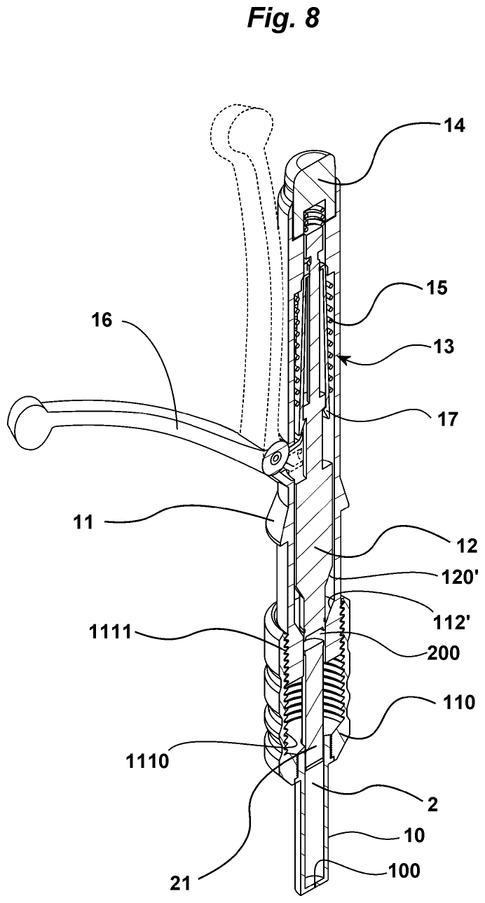

FIGS. 7-10 show a construction variant of the needle-free injection system NIS 1. Here too, matching structural elements may be provided with the same reference symbol. This embodiment too may have a tensioning lever 16 in order to move the piston 12 away from the active substance cartridge 10 and to tension the piston spring 15. FIG. 7 shows the untensioned situation of the piston 12, in which the piston assumes a position in the housing 11 close to the active substance cartridge 10. FIG. 8 shows the tensioned position, in which the tensioning lever 16 has moved the piston 12 into the remote position in relation to the active substance cartridge 10, until the claws which form the piston catch 17 hold the piston 12 in the upper position with the piston spring 15 become tensioned.

After the first injection operation, the actuating piston 12 bears with its lower front face against the upper end face of the injection plunger 21 of the active substance cartridge 10. It should be pointed out that the lower front face of the actuating piston 12 may be concavely formed in order to reliably guide a convex top side of the injection plunger 21.

FIG. 7 shows the set-up after a first injection operation. From the lower position in FIG. 7, the piston 12 may be displaced, by actuation of the tensioning lever 16, into the upper position, which may be seen in FIG. 8. The piston spring 15 may be hereupon tensioned and the piston catch 17 may detain the piston in the upper position.

In FIG. 8, it may be seen that the sleeve-shaped receptacle 110, which bears the active substance cartridge 10, may be provided with an internal thread 1110, which may be screwed onto an external thread 1111 of the housing 11. In order to effect an injection, the sleeve-shaped receptacle 110 for the active substance cartridge 10 may be twisted clockwise with respect to the housing 11, so that the active substance cartridge 10 moves upward and the injection plunger 21 in the active substance cartridge 10 may be brought closer to the actuating piston 12 of the NIS 1. The dosing quantity which may be injected in the next shot may be fixed by the number of revolutions of the receptacle 110 with respect to the housing 11. Once the desired dosing quantity may be set, the nozzle 100 of the active substance cartridge 10 may be placed on the desired skin area and the trigger 14 may be actuated. As a result of the triggering, the piston 12 shoots under the pressure of the piston spring 15 downward against the injection plunger 21, which forces active substance 2 out of the active substance cartridge 10 through the nozzle 100.

The arrows in FIGS. 7, 9 and 10 show respectively the direction in which the piston 12 may be movable.

In the embodiment of FIGS. 7-10, the locking profile and the locking member for limiting the maximum stroke length of the piston 12, and hence the maximum dosing quantity of the active substance, may be formed differently from the previous embodiments. The locking profile 120′ may be formed as a shoulder on a portion of the piston 12 with enlarged diameter. The shoulder may extend from a lower portion of the piston 12 with small diameter obliquely upward and outward along a conical portion, until it reaches a cylindrical portion of the piston 12 with large diameter. That portion of the piston 12 with large diameter may be guided displaceably with small clearance in the housing 11. That portion of the piston 12 with small diameter may be guided with small radial clearance in an opening 200 at the lower end of the housing 11. The frustoconical shoulder, which may form the locking profile 120′, cooperates with a conical pass-through opening in the housing 11, which forms the locking member 112′. In the piston situation represented in FIGS. 7 and 10 with slackened piston spring 15, the frustoconical shoulder of the piston 12 bears substantially flushly against the funnel-shaped piston passage opening 112′.

The injection plunger 21 may be likewise guided with small radial clearance in the opening 200 at the lower end of the housing 11. Because both the lower portion of the piston 12 with small diameter and the injection plunger 21 may be guided with small radial clearance in the opening 200 at the lower end of the housing 11, it may be ensured that the piston 12 presses axially onto the injection plunger 21 and that the injection plunger 21 cannot veer off to the side or break away.

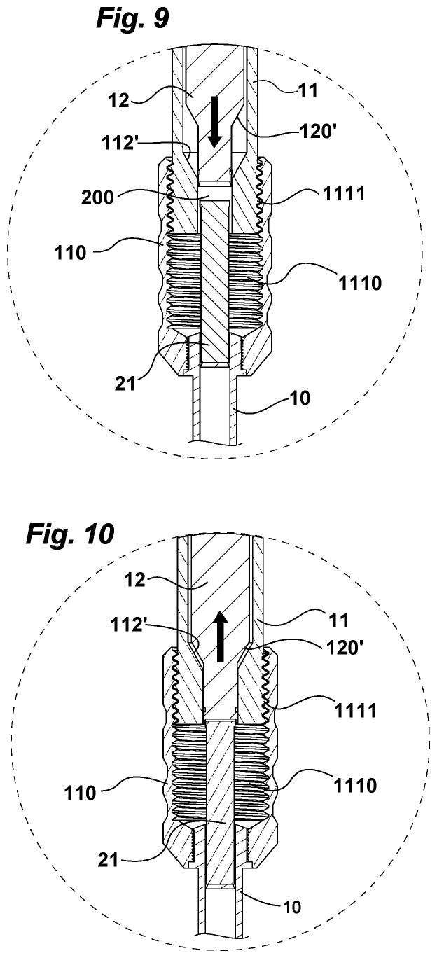

In particular in FIGS. 9 and 10, also the fine threads with which the active substance cartridge 10 may be screwed into the lower end of the sleeve-shaped receptacle 110 of the NIS 1 can be seen.

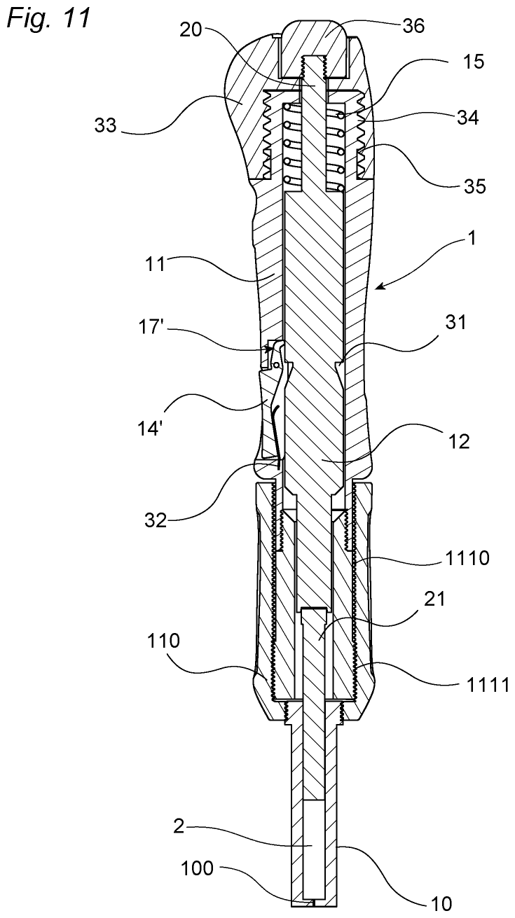

FIGS. 11 to 13 show a further embodiment of the needle-free injection system NIS 1. Here, same components are again provided with same reference symbols. At the end lying opposite the active substance cartridge 10, the housing 11 may have a tensioning thread 34, which may be screwed to an internal thread 35 on a tensioning portion 33. In FIG. 11, the tensioning portion 33 may be screwed tightly onto the rear end of the housing 11. The piston spring 15 may be in the slackened state and forces the piston 12 against the injection plunger 21 of the active substance cartridge 10.

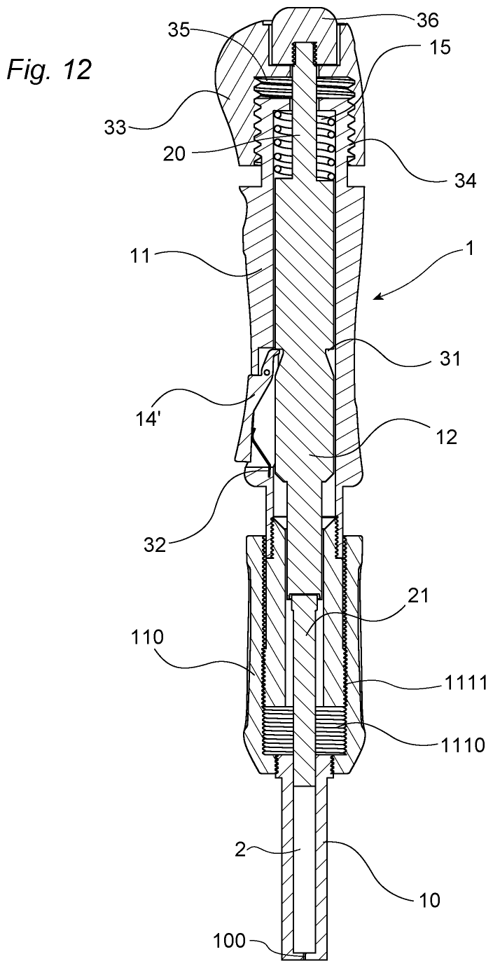

For the tensioning of the piston spring 15, the tensioning portion 33 may be twisted against the rear end of the housing 11. In order to facilitate this, the outer contour of the rear tensioning portion 33 may be non-circularly formed. In FIG. 12, it can be seen that, by twisting of the rear tensioning portion 33 with respect to the housing 11, the piston 12 may be drawn upward via a knob 36 on the rear end of the piston extension 20, and the piston spring 15 may be tensioned. The trigger 14′ may be in this embodiment formed as a pivot switch and may have a latching projection 17′ which forms the piston catch. Once the piston is drawn into the position discernible in FIG. 12, the trigger 14′ may pivot into the latching position, discernible in FIG. 12, in which the piston catch 17′ engages in a groove 31 on the periphery of the piston. A spring plate 32 forces the trigger 14′ into the latching position discernible in FIG. 12.

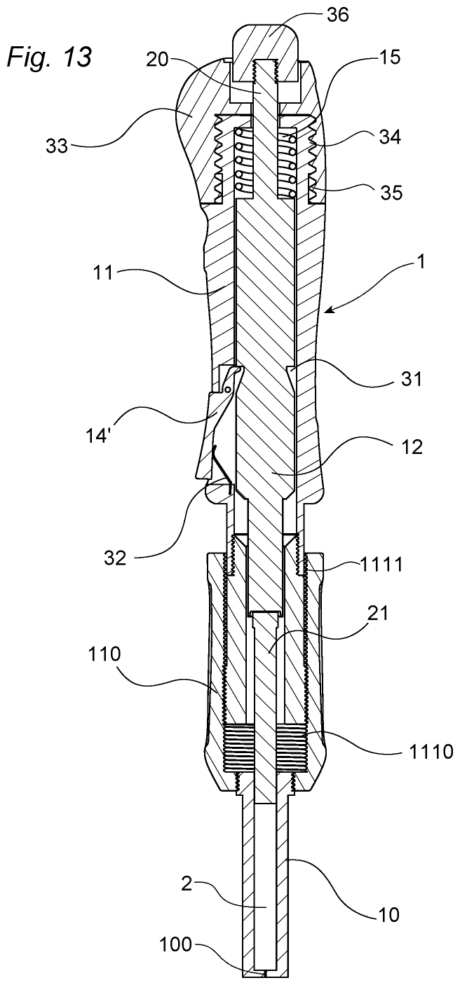

Following the engagement of the piston, the tensioning portion 33 again may be screwed against the housing 11. The knob 36 on the end of the piston extension 20 then may project somewhat to the rear. This configuration may be seen in FIG. 13.

The receptacle 110 now may be twisted, via its internal thread 1110, in relation to the external thread 1111 of the housing 11 in order to vary the distance between the piston 12 and the injection plunger 21 of the active substance cartridge 10 and to thereby fix the quantity of active substance to be injected. The injection or application of the active substance may be realized when a user presses on the trigger 14′, the piston catch (latching projection) 17′ is moved out of the groove 31 of the piston 12, and the axial movement of the piston 12 toward the active substance cartridge 10 is enabled.

The features of the system described herein, in the drawings and in the claims, may be essential for the realization of the system described herein in the various embodiments thereof, both individually and in arbitrary combinations. The system described herein is not restricted to the described embodiments. It may be varied within the scope of the claims, taking into account the knowledge of the relevant person skilled in the art. Other embodiments of the system described herein will be apparent to those skilled in the art from a consideration of the specification and/or an attempt to put into practice the system described herein disclosed herein. It is intended that the specification and examples be considered as illustrative only, with the true scope and spirit of the invention being indicated by the following claims.

1. A needle-free injection system comprising:
an active substance cartridge, which includes a nozzle and a plunger-actuated dosing chamber for pressurized dispensing of an active substance for injection into skin layers;
a housing having a receptacle for a unilaterally reversible arrangement of the active substance cartridge;
a piston, which is movable relative to the housing and is configured to execute a movement for the pressurized dispensing of the active substance from the active substance cartridge;
a driving mechanism for driving the piston, wherein the piston has a starting position relative to the driving mechanism, from which position the piston is released by means of a trigger for executing the movement, wherein the driving mechanism is configured such that the driving mechanism enables a return of the piston into the starting position;
a locking profile; and
a locking member corresponding to the locking profile,
wherein a dispensing quantity of the active substance is adjustable,
wherein a tensioning mechanism for tensioning the driving mechanism is integrated into the injection system,
wherein a receptacle of the active substance cartridge is adjustable in a longitudinal direction by threads in relation to the piston in order to set the dispensing quantity of the active substance,
wherein the locking profile and the locking member define a maximum stroke length of the piston, and accordingly a maximum dispensing quantity of the active agent, so that the maximum dispensing quantity of the active substance is markedly less than a quantity of the content of the active substance cartridge.

2. The needle-free injection system according to claim 1, wherein the maximum dispensing quantity of the active substance is amounts to at most at least one of: 0.1 ml; one-fifth of the capacity of the active substance cartridge.

3. The needle-free injection system according to claim 1, further comprising, for the longitudinal adjustment of the receptacle in relation to the piston;
a threaded joint on the housing, the threaded joint including an internal thread on the receptacle and an external thread on the housing.

4. The needle-free system according to claim 1,
wherein a relationship between the maximum stroke length of the piston and a diameter of the dosing chamber of the active substance cartridge is configured such that the active substance, due to a dispensed quantity thereof, reaches only an upper skin layer and not lower tissue layers.

5. The needle-free injection system according to claim 1,
wherein the locking profile is a collar, which protrudes on the outer periphery of the piston, wherein the locking member is formed as a pass-through opening for the piston, which opening has a lesser diameter than the collar.

6. The needle-free injection system according to claim 1,
wherein the locking profile is a shoulder on the outer periphery of the piston, and wherein the locking member is a complementary stop face that surrounds a pass-through opening for the piston.

7. The needle-free injection system according to claim 6, having at least one of the following features:
the complementary stop face is formed in a shape of a funnel and the shoulder is frustoconically formed;
the piston is guided with small radial clearance in the housing; and
the injection plunger is guided with small radial clearance in the housing.

8. The needle-free injection system according to claim 1,
wherein a pressurization for dispensing the active agent is realized by at least one of the following:
a tensioning spring which spring-loads the piston,
a piston spring,
gas impingement,
an electrically powered pressurizing device.

9. The needle-free injection system according to claim 8,
wherein, the tensioning mechanism includes one of the following:
a tensioning lever, by means of which the tensioning spring can be tensioned several times after an injection in such a way that a removal of the dosing chamber is unnecessary;
an electric motor and a transmission system for tensioning the needle-free injection system, wherein a rotation of the electric motor is transmitted to the transmission system, which effects the movement of the piston in one direction, that creates a tension in the tensioning spring;
a means for raising the piston to generate the necessary tension in the tensioning spring;
a piston extension, wherein the necessary supply of force is realized by rotation of a piston extension;
a tensioning portion, which is screwed onto the housing via a threaded joint and, when twisted with respect to the housing, tensions the tensioning spring.

10. The needle-free injection system according to claim 9,
wherein the tensioning mechanism includes the electric motor and the transmission, wherein the transmission and the electric motor can be decoupled prior to pressing the trigger.

11. The needle-free injection system according to claim 9, wherein the tensioning mechanism includes the piston extension, and
wherein a quantity of active agent dispensed from the active substance cartridge for each individual shot can be set by altering a distance between the piston and the active substance cartridge by rotation of the piston extension.

12. The needle-free injection system according to claim 11, wherein threads are provided along a piston portion to alter the distance.

13. The needle-free injection system according to claim 1, further comprising:
a shank, which is separable from the housing and is fastened to the housing via a thread on its end and a corresponding thread on a portion inside the housing and which is passed through by the piston.

14. The needle-free injection system according to claim 13, further comprising:
a screwing tool, with which the shank can be screwed tightly into the housing and, when necessary, unscrewed therefrom.

15. The needle-free injection system according to claim 1, further comprising:
a plurality of receptacles that are fastenable to the housing, into which active substance cartridges of different size and/or structural form are receivable, wherein a threaded bore at the end of each receptacle corresponds to a specific size of the active substance cartridge.

16. A needle-free injection system comprising:
an active substance cartridge including a plunger-actuated dosing chamber for pressurized dispensing of an active substance for injection into skin layers;
a housing that houses the active substance cartridge;
a piston that moves relative to the housing to cause pressurized dispensing of the active substance from the active substance cartridge;
a locking profile; and
a locking member corresponding to the locking profile,
wherein the locking profile and the locking member define a maximum stroke length of the piston, the maximum stroke length controlling a maximum dispensing quantity of the active substance that is less than a content capacity of the active substance cartridge.

17. The needle-free injection system of claim 16, wherein a receptacle of the active substance cartridge is adjustable in a longitudinal direction by threads in relation to the piston in order to set the dispensing quantity of the active substance.

18. The needle-free injection system of claim 16, wherein the piston has a starting position, and wherein the needle-free injection system further comprises:
a trigger that releases the piston from the starting position to move relative to the housing to cause the pressurized dispensing of the active substance from the active substance.

19. The needle-free injection system of claim 18, further comprising:
a driving mechanism for driving the piston, wherein the driving mechanism enables a return of the piston into the starting position after a pressurized dispensing of the active substance.

20. The needle-free injection system of claim 19, further comprising:
a tensioning mechanism integrated into the injection system for tensioning the driving mechanism.
